(19) United States
(12) Patent Application Publication
(10) Pub. No.: US 2011/0182994 A1
(43) Pub. Date: Jul. 28, 2011

(54) METHODS AND SYSTEMS FOR PRODUCTION OF NANOPARTICLES

(75) Inventors: Julia Ann Kornfield, Pasadena, CA (US); Richard Charles Flagen, Pasadena, CA (US); Bahar Bingol, Istanbul (TR); John Yol Park, Santa Ana, CA (US)

(73) Assignees: S.K. PHARMACEUTICALS, INC., San Juan Capistrano, CA (US); THE CALIFORNIA INSTITUTE OF TECHNOLOGY, Pasadena, CA (US)

(21) Appl. No.: 13/055,918

(22) PCT Filed: Jul. 27, 2009
(86) PCT No.: PCT/US09/51881

§ 371 Date: Apr. 14, 2011

Related U.S. Application Data
(60) Provisional application No. 61/135,940, filed on Jul. 25, 2008.

(51) Int. Cl. A61K 9/14; A61K 31/573; C08J 3/14; B82Y 5/00

(52) U.S. Cl. 424/489; 514/180; 425/10; 264/11; 977/773; 977/915

(57) ABSTRACT

Methods and systems for preparing nanoparticles. A source of a carrier fluid is connected to an inlet of a flow conduit, such as an intravenous solution administration tube with injection ports, such that the carrier fluid flows through the conduit. A substance (e.g., a drug solution or other substance solution) is introduced into the conduit at a first location causing substance nanoparticles to form within and continue to flow thought he conduit. A stabilizer is introduced into the conduit at a second location to cause a stabilizing effect on the nanoparticles. In some embodiments, the stabilizer may limit or deter agglomeration or growth of the nanoparticles, thereby limiting the size of the nanaparticles produced.

### RELATED APPLICATION

This application claims priority to U.S. Provisional Patent Application No. 61/135,940 filed Jul. 25, 2008, the entire disclosure of which is expressly incorporated herein by reference.

### FIELD OF THE INVENTION

The present invention relates to method and apparatus for the production of drug nanoparticles, often smaller than 400 micrometers diameter and suitable for pharmaceutical and opthalmic applications. The method and apparatus is simpler than other methods for synthesis of such particles, and can be implemented using standard, sterile intravenous infusion kits.

### BACKGROUND OF THE INVENTION

Particulate drug delivery systems show considerable problems for delivering drugs to subject, targeting specific organs, tissues, cells, or intracellular compartments, and for influencing residence time of the drug within the circulatory system prior to clearance by the liver or kidneys (WO 2007/150030 A2). The effectiveness of the particular drug delivery system depends strongly on the particle size, composition, and surface chemistry. Control of particle size is critical to the pharmacokinetics of drug delivery. Nanoparticles, i.e., particles with sizes smaller than 1000 nm and often smaller than 100 nm afford special opportunities to engineer the pharmacodynamics of the drug delivery system to achieve particular therapeutic objectives. Although many methods have been developed for drug nanoparticle synthesis, control of particle size remains a challenge. The primary synthesis methods for nanoparticulate drugs are precipitation from solution, emulsion evaporation, salting out, and emulsion evaporation. The nanoprecipitation method is a single-step process wherein a solution containing a substance are mixed with a second fluid in which the substance or the solvent is insoluble or has very low solubility (U.S. Pat. No. 5,118,528). The resulting suspensions of nanoparticles in water are often unstable unless stabilizing agents or surfactants are added to the suspension to minimize transformations in the drug properties after synthesis.

Laboratory techniques been developed to enable synthesis of highly controlled drugs, with considerable effort being invested in recent years into the synthesis of nanoparticles that contain specific therapeutic agents. Particulate drug delivery systems have been developed for delivering a drug to a specific location in the human body by various methods. They have also attracted interest because of the ease with which synthesis parameters can be varied, leading to dramatic variations in the properties of the product nanoparticles. Polymeric nanoparticles have been synthesized from a solution of the polymer in an appropriate solvent such as 1,4-dioxane, tetrahydrofuran, diethylether, acetone, dimethylsulfoxide, acids, alcohols (e.g., methanol, ethanol, isopropanol, etc.). Mixing the polymer solution with a nonsolvent, such as water, induces precipitation to form nanoparticles. If such precipitation is carried out in batch mode, nanoparticles that are produced rapidly aggomerate and settle out from the solution. The solvent may be removed by evaporation. Nanoparticles are recovered by pulverizing the precipitate (U.S. Pat. No. 4,726,955). Similar techniques for preparing nanoparticles for pharmaceutical preparations include wet grinding and milling. The methods for forming nanoparticles by precipitation demonstrate little or no control of particle size and show poor yields, i.e., a relatively low fraction of the therapeutic agent that is fed into the nanoparticle synthesis apparatus is incorporated into nanoparticles in the appropriate size range. Uncontrolled and unpredictable particle size is particularly disadvantageous in the formation of pharmaceutical products since the particle size plays a key role in drug utilization and clearance mechanisms. Furthermore, high throughput production of nanoparticles using the aforementioned techniques can be quite costly. Moreover, many production techniques such as milling and wet grinding introduce the possibility or contamination into the final product. Moreover, the mechanical energy imparted to the therapeutic agent may lead to undesirable alterations in the composition or structure of the therapeutic agent. In short, these methods do not allow for rapid production and screening of particle libraries or economically feasible production of particles. For effective drug therapy, it is desired to deliver sustained and controlled amount of drugs to target tissues and reduce the delivery to non-target tissues to minimize the side effects. Particle characteristics (e.g. composition, size, charge, etc.) can affect the biodistribution and pharmcokinetics of the drug to be delivered. Therefore, it is desirable to control the properties of the nanoparticles to achieve the most effective delivery of a drug.

Agglomeration of product nanoparticles can be minimized by adding a surfactant to the carrier fluid (WO 02/078674). The properties of the initial precipitate are strongly influenced by micromixing (Marchant and David, Experimental evidence for predicting micromixing effects in precipitation, AlChE J. 37: 1698-1710, 1991). A number of approaches have been developed to minimize the mixing time in precipitation systems. Microfluidic systems can reduce mixing times to microseconds by hydrodynamic focusing (Knight, J. B., Vishwanath, A., Brody, J. P. and Austin, R. H. “Hydrodynamic focusing on a silicon chip: Mixing nanoliters in microseconds,” Phys. Rev. Lett. 80: 3863-3866, 1998). A number approaches have been reported for preparing monodisperse nanoparticles in microfluidic devices (deMello and deMello, Lab on a Chip, 4:11N, 2004). Polymeric drug delivery particles have been prepared by nanoprecipitation using controlled mixing of solutions of a block copolymer in an organic solvent with a nonsolvent fluid has been disclosed by Langer et. al. (WO 2007/150030 A2). The resulting nanoparticles are capable of sequestering a drug that is insoluble in water in the hydrophobic core of the resulting nanoparticles. The mixing was achieved by hydrodynamic flow focusing (illustrated in FIG. 1),¹ a well-established technique to control nucleation and growth of particles, which has already been used to grow protein crystals² and to precipitate metal particles in order to deposit metal wires.³ For some applications, laminar interdiffusion does not mix the dissolved precursor sufficiently rapidly to achieve the desired control, so a variety of methods have been devised to accelerate the mixing process by inducing chaotic fluid motions by flowing the cojoined streams in a zig-zag channel (FIG. 2) or through a herringbone channel system. Another approach that has been used to accelerate mixing is to confine the precursor to small droplets to reduce the diffusion distance (FIG. 3). Although the microfluidic devices are capable of producing the desired nanoparticles, fabrication of the microfluidic devices needed for the production of these nanoparticles is complex and expensive. The microfluidic devices used for the preparation of the polymeric nanoparticles in WO 2007/150030 A2 were made of glass or poly(dimethysiloxane) and were fabricated using lithography, which is a complex procedure and requires specialized equipment.

An alternate method for the production of therapeutic nanoparticles is flash precipitation in the impinging jet microreactor described by Johnson and Prud'Homme (WO 02/078674 A1). In this system, illustrated in FIG. 4, the two fluids are introduced into a precipitation chamber through two high velocity jets that impinge against one another. The kinetic energy of the fluid jets is dissipated through chaotic fluctuations that induce rapid mixing. Highly uniform nanoparticles have been synthesized by precipitation in such systems.

While the microfluidic and flash precipitation systems have been demonstrated to produce nanoparticles at useful rates, the systems employed are specialized and somewhat complex. Moreover, the nanoparticle products require additional processing, notably sterilization. The cost and the complexity of the process are among main issues preventing the commercialization of the drug delivery systems based on nanoparticles. Therefore, there is a need for the development of a method for producing nanoparticles in a cost effective way.

### BRIEF SUMMARY OF THE INVENTION

The present invention provides methods and systems for preparing nanoparticles, and nanoparticle compositions prepared thereby.

In accordance with one embodiment of the present invention, a source of a carrier fluid is connected to an inlet of a flow conduit, such as an intravenous solution administration tube with injection ports, such that the carrier fluid flows through the conduit. A substance (e.g., a drug solution or other substance solution) is introduced into the conduit at a first location causing substance nanoparticles to form within and continue to flow thought he conduit. A stabilizer is introduced into the conduit at a second location to cause a stabilizing effect on the nanoparticles. In some embodiments, the stabilizer may limit or deter agglomeration or growth of the nanoparticles, thereby limiting the size of the nanaparticles produced.

In accordance with yet another aspect of the invention, the present invention provides a novel and practical drug nanoparticle synthesis apparatus and method that enables direct synthesis of nanoparticle suspensions of hydrophobic drugs in water. While the system may be applied to block copolymer systems, drug nanoparticles can be synthesized without the addition of the block copolymer. We illustrate the potential of our microfluidic device for rapid nanoparticle synthesis using a steroid (dexamethasone). Dexamethasone has been selected because it is a representative of the diversity of hydrophobic drugs that represent an ongoing challenge for ocular drug delivery. After all eye surgeries, dexamethasone has to be administered in combination with an antibiotic (e.g., oflaxacin) to prevent infection and reduce inflammation.

In accordance with still another aspect of the invention, the present invention provides a method and apparatus for producing nanoparticles of therapeutic agents that are insoluble in water by precipitation of the therapeutic agent from solution in a compatible solvent by mixing . In some embodiments, the apparatus employs readily available, sterile, medical components to minimize the need for specialized fabrication of customized devices for therapeutic nanoparticle synthesis. The apparatus is readily adapted to meet specific needs of a particular therapeutic agent synthesis by allowing new configurations to be developed to enable the use of additional processing stages and other modifications as may be required. Since the components of the apparatus are readily available in prepackaged sterile forms, the production of sterile therapeutic agents can be undertaken without requiring specialized facilities, or specialized training. In general, the apparatus employs standard intravenous infusion sets used in the delivery of intravenous drugs, saline, nutrients, etc. and in blood and plasma transfusions. Slip/Luer adaptors enable rapid and sterile connections of infusion set components. Drug injection is accomplished by insertion of a needle into the septum and delivering a solution of the therapeutic agent in a biocompatible solvent at controlled flow rate into a controlled flow of an antisolvent liquid such as water or sterile saline solution. In some embodiments, mixing of the drug solution into the antisolvent liquid is enhanced by external excitation of the flexible tubing through which the two fluids flow. In some embodiments, said excitation is produced by a sonic toothbrush contacting the exterior of the tubing. In some embodiments, agglomeration of the nanoparticulate drug produced in the precipitation system is quenched by injection of additional water or saline through a second septum to dilute the product particles. In some embodiments, surfactants or other additives may be added through the second septum, or through a third or fourth septum downstream of the nanoparticle synthesis zone.

In some embodiments, the present invention enables point-of-use preparation of suspensions of nanoparticles for immediate use in treatment, thereby eliminating the need for preservatives and stabilizing agents that might create undesirable side effects, and ensuring that the patient receives the nanoparticles in the desired particle size and form.

### BRIEF DESCRIPTION OF THE DRAWINGS

FIG. 1 presents a microfluidic system of the prior art for precipitation of polymeric nanoparticles by interdiffusion of said polymer from into a nonsolvent such as water in laminar flow.

FIG. 2 presents a microfluidic system of the prior art for precipitation of polymeric nanoparticles by interdiffusion of said polymer from into a nonsolvent such as water in a zig-zag channel that induces chaotic flow to accelerate the mixing of the two fluid streams.

FIG. 3 presents a microfluidic system of the prior art for precipitation of polymeric nanoparticles by interdiffusion of said polymer from into a nonsolvent such as water in which the nanoparticles are formed in droplets in a carrier fluid.

FIG. 4 presents jet-mixed reactor of the prior art for flash precipitation of polymeric nanoparticles by interdiffusion of said polymer from into a nonsolvent such as water.

FIG. 5 presents a flow system formed from sterile intravenous infusion kits for precipitation of a drug from solution by interdiffusion into a nonsolvent carrier fluid wherein the drug mixes with the nonsolvent by laminar diffusion.

### DETAILED DESCRIPTION AND EXAMPLES

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

The present invention provides a novel, practical and cost effective apparatus and method for producing drug nanoparticles by nanoparecipitation using controlled mixing of drug solutions in a fluid that is non-solvent for the drug. Rapid mixing and dispersal was achieved by hydrodynamic flow focusing and application of high-frequency mechanical vibration to the drug solution.

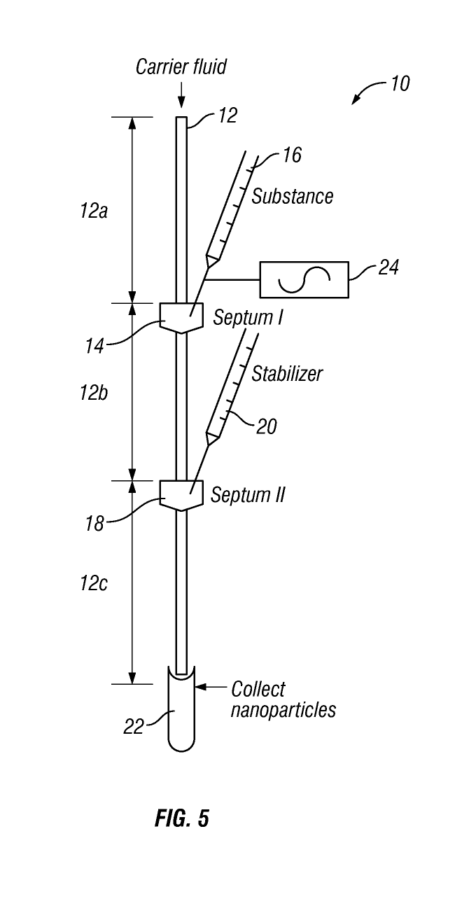

The present invention provides a flow system and method for producing drug nanoparticles. A system according to the present invention may be fabricated, in part, from a sterile medical infusion set or intravenous solution administration set, as shown in FIG. 5. In this example, the system 10 comprises a flow conduit 12 that has an inlet (top) end and an outlet (bottom) end. A first septum or injection port 14 is at a first location on the conduit and a second septum or injection port 18 is at a second location on the conduit, downstream of the first location. A source of carrier fluid (e.g., an aqueous fluid, for example a mixture of saline solution and a surfactant) is connected to the inlet (top) end of the conduit 12. A source of substance 16 (such as a tube, vessel, syringe or syringe pump containing a substance solution) is connected to the first septum or injection port 14 (e.g., by a needle inserted into the injection port) to facilitate introduction of a substance solution into the flow conduit 12 at the first location. A source of stabilizer 20 (such as a tube, vessel, syringe or syringe pump containing an aqueous fluid is connected to the second septum or injection port 18 (e.g., by a needle inserted into the injection port) to facilitate introduction of a stabilizer into the flow conduit 12 at the second location. In typical operation of this system, a carrier fluid (e.g., an aqueoud fluid) flows through a first segment 12a of the conduit 12. At the first location, the substance source 16 delivers the substance (e.g., a drug solution in an organic solvent) into the carrier fluid stream to form a first admixture (i.e., carrier fluid+solvent solution) within which substance-containing nanoparticles form. This first admixture flows through the second segment 12a of the conduit 12. At the second septum or injection port 16, the stabilizer source 20 delivers a stabilizer into the conduit 12. This stabilizer combines with the first admixture to form a second admixture (i.e., carrier fluid+solvent solution (with formed nanoparticles)+stabilizer. The addition of this stabilizer causes a desired effect on the nanoparticles. For example, this stabilizer may deter further agglomeration or growth of the nanoparticles, or my otherwise restrict the size to which the nanoparticles may grow. In this manner, nanoparticles of an optimal size for their intended use may be obtained. Examples of stabilizers that may be used to deter further agglomeration or growth of the nanoparticles, or my otherwise restrict the size to which the nanoparticles may grow include water, aqueous solutions (e.g., saline solutions), aqueous solutions mixed with surfactants, hyaluronic acid solutions (about 0.1% to about 10.0%), polyvinylpyrrolidone (PVP) solutions (about 0.1% to about 10.0%) and cyclodextrin solutions (about 0.1% to about 10.0%).

The nanoparticles are then collected in a vessel 22 at the outlet (bottom) end of the conduit 12 and may be separated from remaining fluids and/or otherwise further processed as desired.

Optionally, in some embodiments of the invention, an ancillary device 24 may be connected to or associated with the system 10 or any component thereof to facilitate the desired nanoparticle formation. For example, such ancillary device 24 may comprise a mixing or motion imparting apparatus (e.g., a mixer, mixing flowpath, vibrator, sonicator, ultrasound apparatus, etc.), one or more pump(s), ultraviolet light sources to deter microbial growth or any other apparatus that may be desirable. The flowrate of each component (carrier fluid, substance and stabilizer) may be controlled in some embodiments by gravity (e.g., by adjusting the height of each fluid source) or injector(s) or pumping apparatus may be used to move the component(s) at desired rates.

The specific embodiment shown in FIG. 5 is an example only. Those of skill in the art will appreciate that other designs and embodiments of this invention may be used. In general, in some embodiments, the system of the present may comprise a flowpath or conduit that has at least two inlets that converge or enter into a common conduit or mixing apparatus. A stream of fluid is capable of flowing through each channel, and streams join and flow into mixing apparatus. One of the streams compromises non-solvent (e.g. aqueous surfactant (e.g. polyoxyethylene sorbitan monooleate (Tween 80) solution), and the other stream compromises a drug solution (e.g. dexamethasone/N-methylpyrrolidone). The flow of carrier fluid into the infusion tube is gravity fed, which can provide a steady and precisely controlled flow rate. Flow rate of the non-solvent for the drug (aqueous surfactant solution) was determined by the height of the column filled with the non-solvent and can be regulated by varying the height of the column filled with the non-solvent. The drug solution is injected into one of the septa of the infusion system using a syringe pump to precisely measure the amount and the rate of injection, which influences the number of nuclei that form and the total size of the drug particles that grow from them. A range of particle sizes were produced with this apparatus due to the time required for the drug to diffuse from the solution into the nonsolvent. a) Sterile, disposable infusion set serves as a microfluidic device. b) The flow of carrier fluid (e.g., sterile saline or artificial tear formulation) into the infusion tube is gravity fed, which can provide a steady and precisely controlled flow rate. The drug solution is injected into one of the septa of the infusion system using a syringe pump to precisely measure the amount and the rate of injection, which influences the number of nuclei that form and the total size of the drug particles that grow from them. High frequency mechanical vibration is applied to the needle to induce rapid dispersal and mixing. In addition to any stabilizing compounds that are included in the carrier fluid and drug solution, precise amount of a composition that envelopes the particles with targeting functional groups and stabilizers.

To induce rapid mixing without the need for the high pressures of the jet-mixed flash precipitation system or the long times required to induce chaotic motion in the zig-zag microfluidic mixer, a mechanical excitation may be applied to the needle that introduces the drug solution into the nonsolvent carrier fluid. The mechanical excitation can be applied with a simple device. In one embodiment, the excitation may be applied using a piezoelectric device. In another embodiment, it may be applied using an electromagnetic oscillator. In another embodiment, it may be applied with an eccentric mass on a rotating shaft. In another embodiment, it may be applied using a “sonic” toothbrush. Other methods for applying the excitation may be applied.

In addition to any stabilizing compounds that are included in the carrier fluid and drug solution, precise amount of a composition that envelopes the particles with targeting functional groups and stabilizers. Additional processing stages can be integrated into the synthesis system so that, after a specified time for growth, the nanoparticles can be diluted to suppress agglomeration, or additional species can be introduced to combine two or more drugs into a single nanoparticle or to functionalize their surfaces to stabilize the nanoparticle suspension. Additional mechanical excitations may be applied to enhance mixing of said additional species with the nanoparticles produced in previous stages. Additional species may also be included in the initial flows of the nonsolvent or of the drug.

The use of this disposable infusion set as our flow system enables rapid exploration of different configurations, and will facilitate production of drug nanoparticles in large quantities to ensure the reasonable production cost.

The operation of our flow system is simple and does not require specialization, knowledge or training in microfluidics.

The flow system provided by this present invention can be useful for engineering particles that have specific characteristics (composition, particle size, etc.). By adjusting any parameter (e.g. flow rate, drug selection, solvent and non-solvent selection, mixing time), particles having specific properties can be engineered.

When invention is employed to prepare nanoparticles for use in pharmaceutical applications a number of criteria may be considered in selecting a suitable solvent. Generally, solvents should have low toxicity so as to provide a final product that is acceptable for administration to a human or animal subject. The solvent may comprise any suitable solvent or mixtures thereof. Examples of specific organic solvents that are useable in this invention include, but are not limited to, organic solvents such as N-methyl pyrrolidone and dimethyl sulfoxide. The non-solvent may be any suitable is an aqueous solution. The nonsolvent compromises water. Solvent or nonsolvent may further compromise surfactants.

Any drug that is more soluble in the drug stream than in the solution in which drug stream is mixed may be used in the microfluidic systems of present invention.

In one preferred embodiment, the sterile flow system may be used to prepare the nanoparticulate suspension at the point of use, thereby eliminating possible degradation of the preparation prior to administration, and eliminating the need for stabilizing agents.

The present invention refers to various patents, published patent applications, journal articles, and other publications, all of which are incorporated herein by reference.

We investigated the ways of rapid dispersal the nuclei in the microfluidic reactor to come up with an efficient process for making nanoparticles. Rapid dispersal of drug solution was achieved by application of high frequency mechanical vibration applied to the drug needle. To evaluate the effect of rapid dispersal of drug solution on the nanoparticle synthesis, we repeated the experiments in the absence of high frequency mechanical vibration as comparison.

The efficiency of each process was determined by quantifying the dexamethasone encapsulated in the particles that are smaller than 450 nm. High performance liquid chromatography coupled with mass spectrometer (HPLC-MS) was used to analyze the samples in terms of dexamethasone concentration. The percent efficiency of each experiment was calculated by determining the concentration of drug before and after filtration with 0.45 μm filter. The percent efficiency is defined as

Percent efficiency=([drug] after filtration −[drug] at saturation limit)/[drug] before filtration)*100

Our results indicate application of high frequency mechanical vibration to the drug needle improves the efficiency of synthesis of nanoparticles that are smaller than 450 nm.

### Specific Example

Flow rate of carrier solution=0.7 mL/s (achieved by filling a tube of 42 cm with carrier solution)

Composition of carrier solution: Polyoxyethylene sorbitan monooleate (Tween 80)/deionized water (5 mg/mL)

Flow rate of drug solution=500 μL/min

Composition of drug solution: Dexamethasone/N-methylpyrrolidone (13.94 wt %)

The efficiency of dexamethasone encapsulation increased from 27% to 40% after sonication of the drug solution.

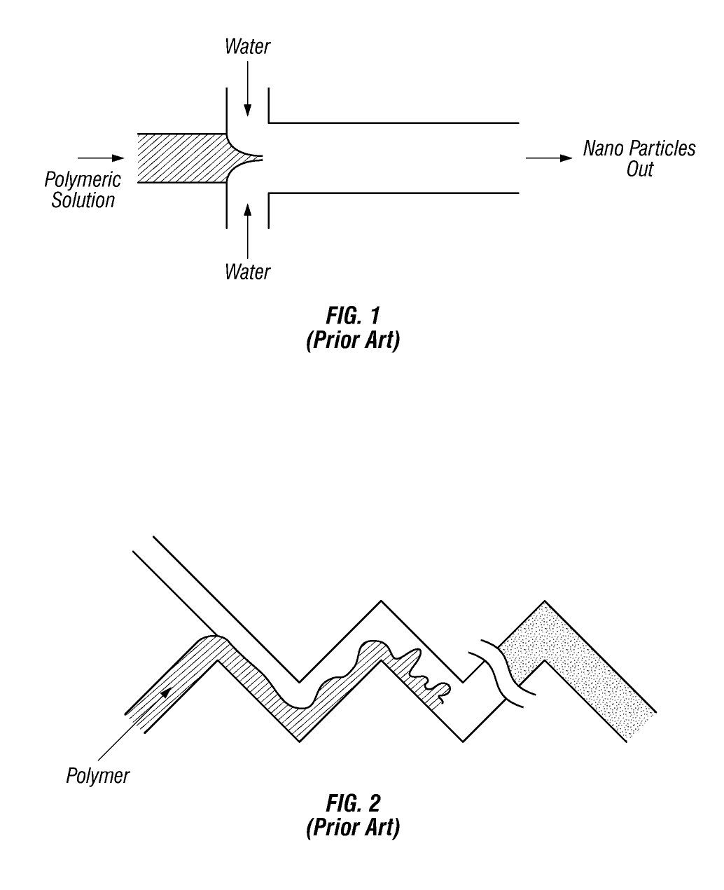

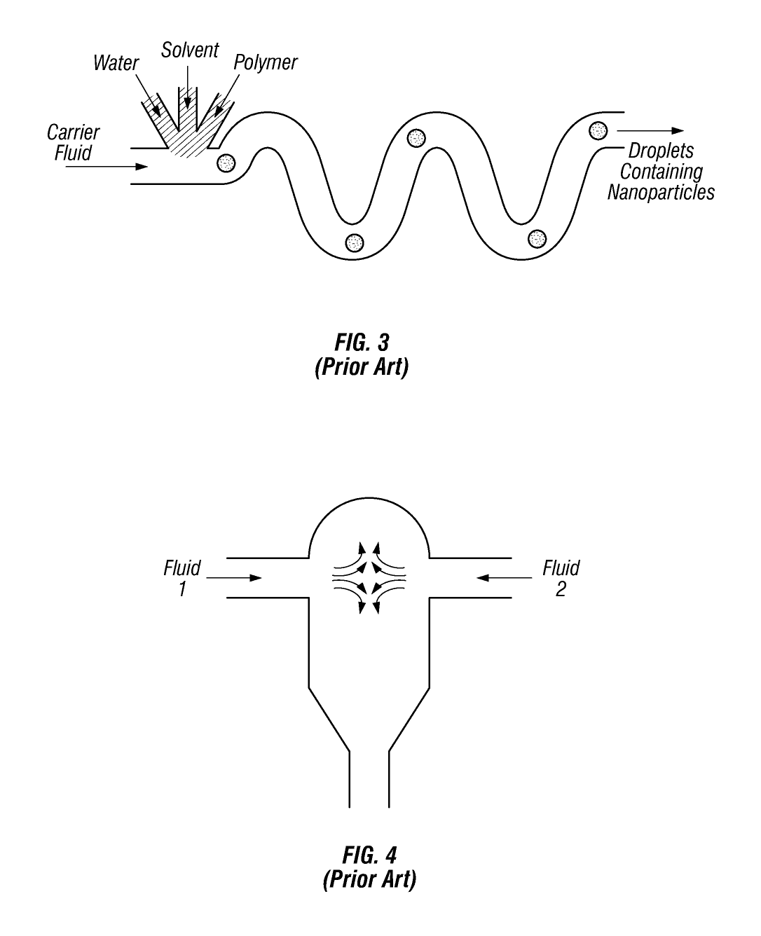

To further improve the efficiency of the nanoparticle preparation process, we applied high frequency mechanical vibration at two different places in our set-up, at the drug needle as well as in the needle in Septum II (FIG. 1b), which was used to introduce non-solvent containing stabilizing compound. Application of additional cavitation in Septum II did not improve the efficiency of nanoparticle synthesis.

To investigate the effect of the temperature of the carrier fluid on the dispersal of the drug solution, we introduced the carrier fluid at 40° C. to the microfluidic device, while we kept the temperature of the drug solution at 21° C. For comparison reasons, we repeated the experiment without heating the carrier fluid. Our results indicate heating the non-solvent enables more homogenous dispersion of the drug nuclei in the carrier fluid, which leads to an increase in the efficiency of the nanoparticle production.

### Specific Example

Flow rate of carrier solution=0.7 mL/s (achieved by filling a tube of 42 cm with carrier solution)

Composition of carrier solution: Polyoxyethylene sorbitan monooleate (Tween 80)/deionized water (1 mg/mL)

Flow rate of drug solution=500 uL/min

Composition of drug solution: Dexamethasone/N-methylpyrrolidone (13.94 wt %)

The efficiency of dexamethasone encapsulation increased from 3% to 13% raising the temperature of the carrier fluid from 25° C. to 40° C.

Our results indicate large quantities of nanoparticle solutions can be produced using the system and method of the present invention, for example a system which employs a medical infusion tube microfluidic device (e.g., a intravenous solution administration set) (˜42 mL/minute) as shown in the example of FIG. 5 and described above.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. All reasonable additions, deletions, modifications and alterations are to be considered equivalents of the described examples and embodiments and are to be included within the scope of the following claims.

What is claimed is:

1. A system for preparing nanoparticles, said system comprising:
a flow conduit having an inlet and an outlet;
a carrier fluid source connected to the inlet such that the carrier fluid flows into the inlet and through the conduit in the direction of the outlet;
a substance source connected to the conduit at a first location between the inlet and outlet such that the substance enters the conduit at the first location, forming a carrier/substance admixture wherein nanoparticles form;
a stabilizer source connected to the conduit at a second location between the first location and the outlet such that at least one stabilizer combines with the substance/solvent admixture and causes at least one stabilizing effect on the nanoparticles.

2. A system according to claim 1 wherein the carrier fluid comprises a non-solvent in which the substance is substantially insoluble.

3. A system according to claim 2 wherein the non-solvent comprises water.

4. A system according to claim 2 wherein the non-solvent comprises an aqueous solution or mixture.

5. A system according to claim 4 wherein the non-solvent comprises an aqueous solution or mixture that contains a surfactant.

6. A system according to claim 5 wherein the non-solvent comprises a mixture of surfactant and water.

7. A system according to claim 1 wherein the substance comprises a solution of a substance in a solvent.

8. A system according to claim 7 wherein the substance comprises a drug.

9. A system according to claim 8 wherein the drug comprises a steroid.

10. A system according to claim 9 wherein the steroid comprises dexamethasone.

11. A system according to claim 7 wherein the solvent is selected from the group consisting of: all organic solvents, N-methylpyrrolidone and dimethyl sulfoxide.

12. A system according to claim 1 wherein the stabilizer comprises an agent that deters agglomeration, deters further enlargement or otherwise restricts the size of the nanoparticles.

13. A system according to claim 12 wherein the stabilizer comprises an aqueous fluid that is delivered in sufficient quantity to deter agglomeration, deters further enlargement or otherwise restricts the size of the nanoparticles.

14. A system according to claim 12 wherein the said nanoparticles are caused to remain smaller than 1000 nm in size.

15. A system according to claim 12 wherein the said nanoparticles are caused to remain smaller than 450 nm in size.

16. A system according to claim 12 wherein the said nanoparticles are caused to remain smaller than 200 nm in size.

17. A system according to claim 12 wherein the said nanoparticles are caused to remain smaller than 100 nm in size.

18. A system according to claim 1 wherein the carrier fluid source provides carrier fluid that is warmed.

19. A method for preparing nanoparticles that comprise a substance, said method comprising the steps of:
(A) obtaining a flow conduit having an inlet and an outlet;
(B) connecting a carrier fluid source to the inlet of the flow conduit and causing carrier fluid to flow into the inlet and through the conduit in the direction of the outlet;
(C) connecting a substance source to the conduit at a first location between the inlet and outlet and causing the substance to enter the conduit at the first location, forming a carrier/substance admixture wherein nanoparticles form;
(D) connecting a stabilizer source to the conduit at a second location between the first location and the outlet and causing at least one stabilizer to enter the conduit at the second location, said at least one stabilizer thereby becoming combined with the substance/solvent admixture and causing at least one stabilizing effect on the nanoparticles.

20. A method according to claim 19 wherein the carrier fluid comprises a non-solvent in which the substance is substantially insoluble.

21. A method according to claim 20 wherein the non-solvent comprises water.

22. A method according to claim 20 wherein the non-solvent comprises an aqueous solution or mixture.

23. A method according to claim 22 wherein the non-solvent comprises an aqueous solution or mixture that contains a surfactant.

24. A method according to claim 23 wherein the non-solvent comprises a mixture of surfactant and water.

25. A method according to claim 19 wherein the substance comprises a solution of a substance in a solvent.

26. A method according to claim 25 wherein the substance comprises a drug.

27. A method according to claim 26 wherein the drug comprises a steroid.

28. A method according to claim 27 wherein the steroid comprises dexamethasone.

29. A method according to claim 25 wherein the solvent comprises an organic solvent.

30. A method according to claim 25 wherein the solvent is selected from the group consisting of: N-methylpyrrolidone and dimethyl sulfoxide.

31. A method according to claim 19 wherein the stabilizer comprises an agent that deters agglomeration of, deters further enlargement of, or otherwise restricts the size of the nanoparticles.

32. A method according to claim 31 wherein the stabilizer comprises an aqueous fluid that is delivered in sufficient quantity to deter agglomeration or further enlargement of the nanoparticles.

33. A method according to claim 31 wherein the said nanoparticles are caused to remain smaller than 1000 nm in size.

34. A system according to claim 31 wherein the said nanoparticles are caused to remain smaller than 450 nm in size.

35. A method according to claim 31 wherein the said nanoparticles are caused to remain smaller than 200 nm in size.

36. A method according to claim 31 wherein the said nanoparticles are caused to remain smaller than 100 nm in size.

37. A method according to claim 19 wherein the carrier fluid source provides carrier fluid that is warmed.

38. A method according to claim 19 wherein the flow conduit comprises an intravenous solution administration tube.

39. A process for preparing nanoparticles by mixing of a pharmaceutical agent or agents or a solution of pharmaceutical agent or agents with a nonsolvent in a branched tubular flow system comprising at least one tube through which said pharmaceutical agent, agents, or solution thereof enters that is cojoined to at least one tube through which said nonsolvent enters to form at least one tube through which the combined flows are discharged after precipitation to form the nanoparticles.

40. The process of claim 39 wherein said branched tubular flow system is formed by assembly of sterile intravenous infusion tubes.

42. The process of claim 39 wherein said drug or drug solution is introduced into said tubular flow system by injection using a hypodermic needle through a septum.

43. The process of claim 39 wherein mixing of said pharmaceutical agent or agents is enhanced by applying mechanical excitation to said flow apparatus.

44. The process of claim 43 wherein mixing of said pharmaceutical agent or agents is enhanced by applying mechanical excitation to said hypodermic needle.

45. The process of claim 39 wherein said the suspension of said nanoparticles in solution is stabilized by addition of stabilizing agents through an additional branch that combines said stabilizing agents with a flow in said system.

46. The process of claim 45 wherein said stabilizing agent branch connects to the flow containing said nanoparticles downstream of the nanoparticle formation region.

47. The process of claim 45 wherein said stabilizing agent branch connects to the nonsolvent flow, thereby mixing said stabilizing agent with said nonsolvent before said nonsolvent flow mixes with said pharmaceutical agent flow.

48. The process of claim 45 wherein said stabilizing agent branch connects to the pharmaceutical agent flow, thereby mixing said stabilizing agent with said pharmaceutical agent before said pharmaceutical agent flow mixes with said nonsolvent flow.

49. The process of claim 39 wherein said nanoparticle production is performed at the point of use for direct administration of said nanoparticles.

50. The process of claim 39 wherein said product nanoparticles are sterilized by filtration downstream of all processing steps.

51. The process of claim 39 wherein said nonsolvent solution is sterile saline.

52. The process of claim 39 wherein said nonsolvent solution is heated to enhance nanoparticle precipitation.

53. The process of claim 39 wherein said flows are fed at a controlled flow rate to said flow system.

54. The process of claim 53 wherein said flows are controlled by adjusting the elevation of the fluid source.

55. The process of claim 53 wherein said flows are driven by gravitational head and controlled using one or more valves.

56. The process of claim 53 wherein said flows are supplied at a controlled rate using a pump.

57. The process of claim 53 wherein said flows are supplied at a controlled rate using a pump.

58. The process of claim 53 wherein said flows are supplied at a controlled rate using a syringe pump.

59. The process of claim 53 wherein said flows are supplied at a controlled rate using a peristaltic pump.

60. The process of claim 19 wherein said nanoparticles are smaller than 1000 nm in size.

61. The process of claim 19 wherein said nanoparticles are smaller than 450 nm in size.

62. The process of claim 19 wherein said nanoparticles are smaller than 200 nm in size.

63. The process of claim 19 wherein said nanoparticles are smaller than 100 nm in size.
